SEQUENCE LISTING (8 sequences)

SEQ ID NO: 1 - protein, 329 aa, Chlamydomonas reinhardtii
  REGION 1..329: note = CrBKT protein
GSGPGIQPTSARPCSRTKHSRFALLAAALTARRVKQFTKQFRSRRMAEDILKLWQRQYHLPREDSDKRTLRERVHLYRPPRSDLGGIAVAVTVIALWATLFVYGLWFVKLPWALKVGETATSWATIAAVFFSLEFLYTGLFITTHDAMHGTIALRNRRLNDFLGQLAISLYAWFDYSVLHRKHWEHHNHTGEPRVDPDFHRGNPNLAVWFAQFMVSYMTLSQFLKIAVWSNLLLLAGAPLANQLLFMTAAPILSAFRLFYYGTYVPHHPEKGHTGAMPWQVSRTSSASRLQSFLTCYHFDLHWEHHRWPYAPWWELPKCRQIARGAALA

SEQ ID NO: 2 - protein, 574 aa, synthetic construct
  REGION 1..574: note = CrBKT_YFP
GSGPGIQPTSARPCSRTKHSRFALLAAALTARRVKQFTKQFRSRRMAEDILKLWQRQYHLPREDSDKRTLRERVHLYRPPRSDLGGIAVAVTVIALWATLFVYGLWFVKLPWALKVGETATSWATIAAVFFSLEFLYTGLFITTHDAMHGTIALRNRRLNDFLGQLAISLYAWFDYSVLHRKHWEHHNHTGEPRVDPDFHRGNPNLAVWFAQFMVSYMTLSQFLKIAVWSNLLLLAGAPLANQLLFMTAAPILSAFRLFYYGTYVPHHPEKGHTGAMPWQVSRTSSASRLQSFLTCYHFDLHWEHHRWPYAPWWELPKCRQIARGAALAGSGRSDVVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 3 - protein, 611 aa, synthetic construct
  REGION 1..611: note = CrBKT_YFP + PsaD
MAVMMRTQAPAATRASSRVAVAARPAARRAVVVRAEAGSGPGIQPTSARPCSRTKHSRFALLAAALTARRVKQFTKQFRSRRMAEDILKLWQRQYHLPREDSDKRTLRERVHLYRPPRSDLGGIAVAVTVIALWATLFVYGLWFVKLPWALKVGETATSWATIAAVFFSLEFLYTGLFITTHDAMHGTIALRNRRLNDFLGQLAISLYAWFDYSVLHRKHWEHHNHTGEPRVDPDFHRGNPNLAVWFAQFMVSYMTLSQFLKIAVWSNLLLLAGAPLANQLLFMTAAPILSAFRLFYYGTYVPHHPEKGHTGAMPWQVSRTSSASRLQSFLTCYHFDLHWEHHRWPYAPWWELPKCRQIARGAALAGSGRSDVVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 4 - protein, 627 aa, synthetic construct
  REGION 1..627: note = CrBKT_YFP + PsaD + Strep-tag II
MAVMMRTQAPAATRASSRVAVAARPAARRAVVVRAEAGSGPGIQPTSARPCSRTKHSRFALLAAALTARRVKQFTKQFRSRRMAEDILKLWQRQYHLPREDSDKRTLRERVHLYRPPRSDLGGIAVAVTVIALWATLFVYGLWFVKLPWALKVGETATSWATIAAVFFSLEFLYTGLFITTHDAMHGTIALRNRRLNDFLGQLAISLYAWFDYSVLHRKHWEHHNHTGEPRVDPDFHRGNPNLAVWFAQFMVSYMTLSQFLKIAVWSNLLLLAGAPLANQLLFMTAAPILSAFRLFYYGTYVPHHPEKGHTGAMPWQVSRTSSASRLQSFLTCYHFDLHWEHHRWPYAPWWELPKCRQIARGAALAGSGRSDVVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKIEGRDIEFWSHPQFEK

SEQ ID NO: 5 - DNA, 1272 bp, Chlamydomonas reinhardtii
  misc_feature 1..1272: note = CrBKT sequence
  source 1..1272: mol_type = other DNA
ggccccggcatccagcccaccagcgcccgcccctgcagccgcaccaagcacagccgcttcgccctgctggccgccgccctgaccgcccgccgcgtgaagcagttcaccaagcagttccgcagccgccgcatggccgaggacatcctgaagctgtggcagcgccagtaccacctgccccgcgaggacagcgacaagcgcaccctgcgcgagcgcgtgcacctgtaccgccccccccgcagcgacctgggcggcatcgccgtggccgtgaccgtgatcgccctgtgggccaccctgttcgtgtacggcctgtggttcgtgaagctgccctgggccctgaaggtggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcagggcgagaccgccaccagctgggccaccatcgccgccgtgttcttcagcctggagttcctgtacaccggcctgttcatcaccacccacgacgccatgcacggcaccatcgccctgcgcaaccgccgcctgaacgacttcctgggccagctggccatcagcctgtacgcctggttcgactacagcgtgctgcaccgcaagcactgggagcaccacaaccacaccggcgagccccgcgtggaccccgacttccaccgcggcaaccccaacctggccgtgtggttcgcccagttcatggtgagctacatgaccctgagccagttcctgaagatcgccgtgtggagcaacctgctgctgctggccggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggcgcccccctggccaaccagctgctgttcatgaccgccgcccccatcctgagcgccttccgcctgttctactacggcacctacgtgccccaccaccccgagaagggccacaccggcgccatgccctggcaggtgagccgcaccagcagcgccagccgcctgcagagcttcctgacctgctaccacttcgacctgcactgggagcaccaccgctggccctacgccccctggtgggagctgcccaagtgccgccagatcgcccgcggcgccgccctggccg

SEQ ID NO: 6 - DNA, 2335 bp, synthetic construct
  misc_feature 1..2335: note = CrBKT_YFP sequence
  source 1..2335: mol_type = other DNA
ggccccggcatccagcccaccagcgcccgcccctgcagccgcaccaagcacagccgcttcgccctgctggccgccgccctgaccgcccgccgcgtgaagcagttcaccaagcagttccgcagccgccgcatggccgaggacatcctgaagctgtggcagcgccagtaccacctgccccgcgaggacagcgacaagcgcaccctgcgcgagcgcgtgcacctgtaccgccccccccgcagcgacctgggcggcatcgccgtggccgtgaccgtgatcgccctgtgggccaccctgttcgtgtacggcctgtggttcgtgaagctgccctgggccctgaaggtggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcagggcgagaccgccaccagctgggccaccatcgccgccgtgttcttcagcctggagttcctgtacaccggcctgttcatcaccacccacgacgccatgcacggcaccatcgccctgcgcaaccgccgcctgaacgacttcctgggccagctggccatcagcctgtacgcctggttcgactacagcgtgctgcaccgcaagcactgggagcaccacaaccacaccggcgagccccgcgtggaccccgacttccaccgcggcaaccccaacctggccgtgtggttcgcccagttcatggtgagctacatgaccctgagccagttcctgaagatcgccgtgtggagcaacctgctgctgctggccggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggcgcccccctggccaaccagctgctgttcatgaccgccgcccccatcctgagcgccttccgcctgttctactacggcacctacgtgccccaccaccccgagaagggccacaccggcgccatgccctggcaggtgagccgcaccagcagcgccagccgcctgcagagcttcctgacctgctaccacttcgacctgcactgggagcaccaccgctggccctacgccccctggtgggagctgcccaagtgccgccagatcgcccgcggcgccgccctggccggcagcggcagatctgacgtcgtgagcaagggcgaggagctgttcaccggcgtggtgcccatcctggtggagctggacggcgacgtgaacggccacaagttcagcgtgagcggcgagggcgagggcgacgccacctacggcaagctgaccctgaagctgatctgcaccaccggcaagctgcccgtgccctggcccaccctggtgaccaccctgggctacggcctgcagtgcttcgcccgctaccccgaccacatgaagcagcacgacttcttcaagagcgccatgcccgagggctacgtgcaggagcgcaccatcttcttcaaggacgacggtgagcttgcggggttgcgagcaacactccagcaacgaacagtgcccaagtcaggaatctgcagtcagcctgggctttcggcggctttttcttgggcaaacagcttgcactcatgccagcgcggcttgtccagcctcacttgagctttccagctgctaccagccgggctatacgacagcgacagagccatagcgtggaatcacttatttgggttgccgaagtagcggtcggagcgtgagttcttggtcaagccgccccttatccggttcctgtccgtgtctttgtccctcgttcacccttcgcggcacccttcatccccttgcttgcaggtaactacaagacccgcgccgaggtgaagttcgagggcgacaccctggtgaaccgcatcgagctgaagggcatcgacttcaaggaggacggcaacatcctgggccacaagctggagtacaactacaacagccacaacgtgtacatcaccgccgacaagcagaagaacggcatcaaggccaacttcaagatccgccacaacatcgaggacggcggcgtgcagctggccgaccactaccagcagaacacccccatcggcgacggccccgtgctgctgcccgacaaccactacctgagctaccagagcaagctgagcaaggaccccaacgagaagcgcgaccacatggtgctgctggagttcgtgaccgccgccggcatcaccctgggcatggacgagctgtacaag

SEQ ID NO: 7 - DNA, 2452 bp, synthetic construct
  misc_feature 1..2452: note = CrBKT_YFP + PsaD sequence
  source 1..2452: mol_type = other DNA
atggccgtcatgatgcgcacccaggcgcccgctgccactcgcgcttcatcgcgcgtcgctgttgccgctcgcccggctgctcgccgcgccgtggtggtccgcgccgaggctggatccggccccggcatccagcccaccagcgcccgcccctgcagccgcaccaagcacagccgcttcgccctgctggccgccgccctgaccgcccgccgcgtgaagcagttcaccaagcagttccgcagccgccgcatggccgaggacatcctgaagctgtggcagcgccagtaccacctgccccgcgaggacagcgacaagcgcaccctgcgcgagcgcgtgcacctgtaccgccccccccgcagcgacctgggcggcatcgccgtggccgtgaccgtgatcgccctgtgggccaccctgttcgtgtacggcctgtggttcgtgaagctgccctgggccctgaaggtggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcagggcgagaccgccaccagctgggccaccatcgccgccgtgttcttcagcctggagttcctgtacaccggcctgttcatcaccacccacgacgccatgcacggcaccatcgccctgcgcaaccgccgcctgaacgacttcctgggccagctggccatcagcctgtacgcctggttcgactacagcgtgctgcaccgcaagcactgggagcaccacaaccacaccggcgagccccgcgtggaccccgacttccaccgcggcaaccccaacctggccgtgtggttcgcccagttcatggtgagctacatgaccctgagccagttcctgaagatcgccgtgtggagcaacctgctgctgctggccggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggcgcccccctggccaaccagctgctgttcatgaccgccgcccccatcctgagcgccttccgcctgttctactacggcacctacgtgccccaccaccccgagaagggccacaccggcgccatgccctggcaggtgagccgcaccagcagcgccagccgcctgcagagcttcctgacctgctaccacttcgacctgcactgggagcaccaccgctggccctacgccccctggtgggagctgcccaagtgccgccagatcgcccgcggcgccgccctggccggcagcggcagatctgacgtcgtgagcaagggcgaggagctgttcaccggcgtggtgcccatcctggtggagctggacggcgacgtgaacggccacaagttcagcgtgagcggcgagggcgagggcgacgccacctacggcaagctgaccctgaagctgatctgcaccaccggcaagctgcccgtgccctggcccaccctggtgaccaccctgggctacggcctgcagtgcttcgcccgctaccccgaccacatgaagcagcacgacttcttcaagagcgccatgcccgagggctacgtgcaggagcgcaccatcttcttcaaggacgacggtgagcttgcggggttgcgagcaacactccagcaacgaacagtgcccaagtcaggaatctgcagtcagcctgggctttcggcggctttttcttgggcaaacagcttgcactcatgccagcgcggcttgtccagcctcacttgagctttccagctgctaccagccgggctatacgacagcgacagagccatagcgtggaatcacttatttgggttgccgaagtagcggtcggagcgtgagttcttggtcaagccgccccttatccggttcctgtccgtgtctttgtccctcgttcacccttcgcggcacccttcatccccttgcttgcaggtaactacaagacccgcgccgaggtgaagttcgagggcgacaccctggtgaaccgcatcgagctgaagggcatcgacttcaaggaggacggcaacatcctgggccacaagctggagtacaactacaacagccacaacgtgtacatcaccgccgacaagcagaagaacggcatcaaggccaacttcaagatccgccacaacatcgaggacggcggcgtgcagctggccgaccactaccagcagaacacccccatcggcgacggccccgtgctgctgcccgacaaccactacctgagctaccagagcaagctgagcaaggaccccaacgagaagcgcgaccacatggtgctgctggagttcgtgaccgccgccggcatcaccctgggcatggacgagctgtacaag

SEQ ID NO: 8 - DNA, 2503 bp, synthetic construct
  misc_feature 1..2503: note = CrBKT_YFP PsaD + Strep-tag II gene sequence
  source 1..2503: mol_type = other DNA
atggccgtcatgatgcgcacccaggcgcccgctgccactcgcgcttcatcgcgcgtcgctgttgccgctcgcccggctgctcgccgcgccgtggtggtccgcgccgaggctggatccggccccggcatccagcccaccagcgcccgcccctgcagccgcaccaagcacagccgcttcgccctgctggccgccgccctgaccgcccgccgcgtgaagcagttcaccaagcagttccgcagccgccgcatggccgaggacatcctgaagctgtggcagcgccagtaccacctgccccgcgaggacagcgacaagcgcaccctgcgcgagcgcgtgcacctgtaccgccccccccgcagcgacctgggcggcatcgccgtggccgtgaccgtgatcgccctgtgggccaccctgttcgtgtacggcctgtggttcgtgaagctgccctgggccctgaaggtggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcagggcgagaccgccaccagctgggccaccatcgccgccgtgttcttcagcctggagttcctgtacaccggcctgttcatcaccacccacgacgccatgcacggcaccatcgccctgcgcaaccgccgcctgaacgacttcctgggccagctggccatcagcctgtacgcctggttcgactacagcgtgctgcaccgcaagcactgggagcaccacaaccacaccggcgagccccgcgtggaccccgacttccaccgcggcaaccccaacctggccgtgtggttcgcccagttcatggtgagctacatgaccctgagccagttcctgaagatcgccgtgtggagcaacctgctgctgctggccggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggcgcccccctggccaaccagctgctgttcatgaccgccgcccccatcctgagcgccttccgcctgttctactacggcacctacgtgccccaccaccccgagaagggccacaccggcgccatgccctggcaggtgagccgcaccagcagcgccagccgcctgcagagcttcctgacctgctaccacttcgacctgcactgggagcaccaccgctggccctacgccccctggtgggagctgcccaagtgccgccagatcgcccgcggcgccgccctggccggcagcggcagatctgacgtcgtgagcaagggcgaggagctgttcaccggcgtggtgcccatcctggtggagctggacggcgacgtgaacggccacaagttcagcgtgagcggcgagggcgagggcgacgccacctacggcaagctgaccctgaagctgatctgcaccaccggcaagctgcccgtgccctggcccaccctggtgaccaccctgggctacggcctgcagtgcttcgcccgctaccccgaccacatgaagcagcacgacttcttcaagagcgccatgcccgagggctacgtgcaggagcgcaccatcttcttcaaggacgacggtgagcttgcggggttgcgagcaacactccagcaacgaacagtgcccaagtcaggaatctgcagtcagcctgggctttcggcggctttttcttgggcaaacagcttgcactcatgccagcgcggcttgtccagcctcacttgagctttccagctgctaccagccgggctatacgacagcgacagagccatagcgtggaatcacttatttgggttgccgaagtagcggtcggagcgtgagttcttggtcaagccgccccttatccggttcctgtccgtgtctttgtccctcgttcacccttcgcggcacccttcatccccttgcttgcaggtaactacaagacccgcgccgaggtgaagttcgagggcgacaccctggtgaaccgcatcgagctgaagggcatcgacttcaaggaggacggcaacatcctgggccacaagctggagtacaactacaacagccacaacgtgtacatcaccgccgacaagcagaagaacggcatcaaggccaacttcaagatccgccacaacatcgaggacggcggcgtgcagctggccgaccactaccagcagaacacccccatcggcgacggccccgtgctgctgcccgacaaccactacctgagctaccagagcaagctgagcaaggaccccaacgagaagcgcgaccacatggtgctgctggagttcgtgaccgccgccggcatcaccctgggcatggacgagctgtacaagatcgagggcagggatatcgaattctggagccacccgcagttcgagaagtaa

(19) United States
(12) Patent Application Publication
(10) Pub. No.: US 2025/0243527 A1
(43) Pub. Date: Jul. 31, 2025

(54) USE OF A MODIFIED B-CAROTENE KETOLASE (BKT) OR A CORRESPONDING NUCLEIC ACID FOR IMPROVING RESISTANCE TO OXIDATIVE STRESS AND/OR PHOTOINHIBITION OF HOST ORGANISMS, IMPROVING BIOMASS PRODUCTIVITY OF HOST ORGANISMS AND/OR PREVAILING OVER OTHER COMPETING ORGANISMS UPON CULTIVATION IN HIGH LIGHT CONDITIONS

(71) Applicant: UNIVERSITA' DEGLI STUDI DI VERONA, Verona (IT)

(72) Inventors: Matteo Ballottari, Verona (IT); Stefano Cazzaniga, Verona (IT); Federico Perozeni, Verona (IT); Nico Betterle, Verona (IT)

(21) Appl. No.: 18/705,038

(22) PCT Filed: Oct. 28, 2022
(86) PCT No.: PCT/IB2022/060381

§ 371 Date: Apr. 26, 2024

(30) Foreign Application Priority Data
Oct. 29, 2021 (IT) 102021000027824

(51) Int. Cl. C12P 23/00; C12N 9/02; C12N 15/82

(52) U.S. Cl. CPC C12P 23/00; C12N 9/0077; C12N 9/0083; C12N 15/825; C12N 15/8271

(57) ABSTRACT

There is disclosed a use of a modified β-carotene ketolase, a corresponding gene and a microalgae strain comprising the same for improving the resistance to oxidative stress and/or photoinhibition of host organisms or for improving biomass productivity of host organisms and/or prevailing over other competing organisms upon cultivation in high light conditions.

### CROSS-REFERENCE TO RELATED APPLICATIONS

This Patent application claims priority from Italian Patent Application No. 102021000027824 filed on Oct. 29, 2021, the entire disclosure of which is incorporated herein by reference.

### TECHNICAL FIELD OF THE INVENTION

The present invention relates to the use of a modified β-carotene ketolase (BKT) or a corresponding nucleic acid for improving the resistance to oxidative stress and/or photoinhibition of host organisms or for improving biomass productivity of host organisms and/or prevailing over other competing organisms upon cultivation in high light conditions.

### BACKGROUND ART

Photosynthetic organisms take advantage of the almost infinite supply of sunlight that reaches our planet to assimilate CO₂ into organic molecules and accumulate biomass. This biomass is processed into feedstock, food and biofuels or used to obtain a huge array of complex chemicals with many different applications ranging from fertilizers to drugs.

Microalgae are a wide group of eukaryotic photosynthetic unicellular organisms having potential production yield far higher than crops plants (Stephenson et al., 2011). Even if only eukaryotic organisms should be classified among microalgae from a taxonomic point of view, usually prokaryotic photosynthetic species are also considered among microalgae. Thanks to their simpler unicellular structure, all their biomass is photosynthetically active and they have an easier access to light, CO₂ and nutrients. Microalgae grow fast and, in optimal condition, they have a doubling time below one day while terrestrial plants can only reach up a few harvest cycles per year. They are metabolically flexible and can shift from autotrophic grow to mixotrophic depending on the growth condition and substrate used. Besides, microalgae include species that adapt to all the different atmospheric conditions of the planet and are resilient to many different types of stress like extreme pH, salinity low and high temperature (Guiry, 2012). Thanks to this natural variability, they do not compete with resources for conventional food production and can grow using brackish or wastewater or sea water for marine species. Several microalgal species are nowadays cultivated in simple or more complex artificial cultivation system, as open ponds or closed photobioreactors.

The combination of the light energy-capturing ability of photosynthesis with the high yields of controlled microbial cultivation make microalgae potentially valuable organisms for economical, industrial-scale production processes in areas including nutrition, aquaculture, pharmaceuticals, and biofuels. Several microalgal species were indeed included among the possible “novel food” sources for human consumption (Bernaerts et al., 2019, Koyande et al., 2019a) and some strains are nowadays considered for high-value metabolites to be used in the cosmetic sector, for fish/animal feed, for the production of bioplastics, drugs, or other commodities (Camacho et al., 2019, Koyande et al., 2019b, Sathasivam et al., 2019, Freudenberg et al., 2020, Petroutsos et al., 2021). Microalgae also have a high potential for environmental applications, being used as biostimulants, fertilizers and/or biopesticides in agriculture, reducing the negative environmental impact of fertilizers and pesticides (Mutale-Joan et al., 2020). Finally, the potential use of microalgae biomass for biofuels has been considered.

Despite their potential, the industrial application of microalgae is nowadays limited to production of high value bioactive compounds and recombinant proteins because of a series of physiological and technical constrains that limit the economic feasibility of the process (Borowitzka, 2013, Quinn and Davis, 2015). Improving microalgae cultivation is a multivariable problem that regards culturing techniques, applications and economics but, first of all, it needs an improvement of the sunlight utilization.

The model organism for green algae is Chlamydomonas reinhardtii as its genome has already been sequenced and characterized. C. reinhardtii is characterized by a ˜10 μm cell, two flagella and a large chloroplast.

C. reinhardtii, however, is rarely used at the industrial level, where more robust and fast-growing species are preferred, as for instance species belonging to Chlorella, Scenedesmus, Nannochloropsis genus, among others. However, C. reinhardtii is the species for eukaryotic microalgae where the biotechnological tool for genetic engineering are more developed, among which CRISPR-genome editing, synthetic biology, gene overexpression, etc. (Ng et al., 2017, Crozet et al., 2018, Lin et al., 2019, Baier et al., 2020). Recently a method for obtaining high cell density cultivation of C. reinhardtii has been reported based on optimization of the growth medium paving the way for possible industrial application even of this species (Freudenberg et al., 2021).

Annually photosynthesis results in the assimilation of upwards of 100 petagrams (Pg) of carbon (Field et al., 1998). Oxygenic photosynthesis is carried out by four multi-subunit membrane-protein complexes in the thylakoid membrane: two photosystems (PSI and PSII), cytochrome b6f and ATPase (Nelson and Ben Shem, 2004). Each photosystem is composed by a core complex connected to an array of protein subunits called antennae complexes that increase light absorption (Van Amerongen and Croce, 2013). Both antenna and core complexes include several protein subunits that binds pigments chlorophylls a, b and carotenoids.

Carotenoids, pigments ranging from light yellow to deep red, are present in all photosynthetic organisms, where they play a crucial role in photosynthesis. They are involved in photosystem assembly and light harvesting in the Photosynthetic Active Region (PAR) where chlorophyll absorbance is weak. Carotenoids contribute to the antioxidant network of the chloroplast and detoxify ROS generated by photosynthesis preventing lipid peroxidation. They are considered the most potent quenchers of singlet oxygen and can react with any of the radical species encountered in the biological system such as hydrogen peroxide, singlet oxygen, nitrogen oxides and super oxide anion (Paiva and Russell, 1999). Carotenoids, bound to photosynthetic subunits, are in close contact with chlorophyll molecules and they are involved in several photoprotective mechanisms to reduce the risk of photoinhibition and reactive oxygen species formation (Peterman et al., 1995).

Carotenoids are found in chloroplasts of photosynthetic organisms and in chromoplasts in fruits and flowers (Britton et al., 1998). Some carotenoids like astaxanthin (3,3′-dihydroxy-β, β-carotene-4,4′-dione) from Haematococcus lacustria (formely H. pluvialis) could be also found in the cytosol. Carotenoids occur as free form in chloroplast and leaves and are normally esterified in other locations. Carotenoids are derived from a 40-carbon polyene chain that is the backbone of the molecule that terminates with cyclic end-groups (rings) and could be complemented with oxygen-containing functional groups. Based on their chemical structure, carotenoids are classified into two groups: hydrocarbons cyclized at both ends commonly known as carotenes and xanthophylls, the oxygenated derivatives of these hydrocarbons. The nature of the specific end groups in carotenoids influence their optical property and their polarity, changing the way in which individual carotenoids interact with biological membranes. The fundamental step in the carotenoid biosynthesis is the condensation of two Geranylgeranyl diphosphate (GGPP) to form colourless C-40 compound phytoene. Phytoene undergoes a series of four desaturation by phyotene desaturae (PDS) to form lycopene. The desaturation reactions increase the conjugated series carbon-carbon double bonds that give carotenoids constitutes their chromophore property and transform the phytoene into the pink-coloured lycopene. Cyclization of lycopene by lycopene β- and ϵ-cyclase produce β-carotene, with a β-ionone ring at each end or α-carotene, with one ϵ-ring and one β-ring. β-carotene is bound to the reaction centres of PSI and PSII and to the antenna of PSI while α-carotene is normally rapidly converted in xanthophylls and present only in traces. The different oxygen groups added to carotenes in different positions of the rings and their different combinations can produce hundreds of different xanthophylls but only a limited group on then is used in photosynthesis. In the model organism for green algae, Chlamydomonas reinhardtii, these xanthophylls are ε-β lutein and loroxanthin and β-β zeaxanthin, violaxanthin and neoxanthin (Niyogi et al., 1997, Perozeni et al., 2020b). Zeaxanthin and lutein are produced by hydroxylation at the C-3 position of each ring of β-carotene and α-carotene respectively. In low light condition a part of lutein is converted into loroxanthin (Takaichi, 2011a). Under control light conditions zeaxanthin is readily converted to violaxanthin by the introduction of 5, 6-epoxy groups into β-rings, a reaction catalysed by the enzyme zeaxanthin epoxidase (ZEP). In high light, violaxanthin deepoxidase (VDE) catalyzes de-epoxidation reaction that convert violaxanthin back to zeaxanthin. This interconversion of zeaxanthin and violaxanthin is named xanthophyll cycle, is a key for adaptation to changing environmental conditions and for NPQ activation (Demmig-Adams et al., 1996). In the last step of the β-β pathway, violaxanthin is transformed into neoxanthin by the activity of neoxanthin synthase (NSY). The stoichiometry of different xanthophylls and their specific binding site in light harvesting complexes is also conserved in a wide range of taxa from microalgae to crops plants indicating a specific function of the different carotenoids (Cunningham and Gantt, 1998, Takaichi, 2011b, Girolomoni et al., 2020). Despite this conserved distribution, in other species of algae different types of xanthophylls are present. Diatoms and multicellular brown algae accumulate xanthophylls fucoxanthin, diadinoxanthin and diatoxanthin (Bertrand, 2010) while some species of microalgae are able to accumulate ketocarotenoids like astaxanthin (Shah et al., 2016, Roth et al., 2017, Novoveská et al., 2019). Ketocarotenoids show a higher antioxidant capacity with respect to the carotenes and xanthophylls normally accumulated in green microalgae (Lemoine and Schoefs, 2010, Perozeni et al., 2020b). They are obtained by enzyme β-carotene ketolase (BKT) that catalyses the addition of a keto-group at the C4 position of carotenoids β-rings. The most studied ketocarotenoid is astaxanthin that is synthetized adding a keto-group on both rings of zeaxanthin; thanks to this carbonyl group this pigment have an antioxidant activity 10 times stronger than zeaxanthin and β-carotene (Miki, 1991, Krinsky, 1993). Higher plants and most microalgae do not possess the carotene ketolase activity consequently do not synthesize ketocarotenoids. The most prominent source of astaxanthin is fresh-water microalgae Haematococcus lacustris that accumulate this ketocarotenoid up to 5% of its dry weight. Astaxanthin is classified as secondary carotenoid because unlike primary xanthophylls, that are structural and functional components of the photosynthetic apparatus, it is usually produced in high quantity only after specific environmental stimuli. H. lacustris do not accumulate astaxanthin in all its life cycle but only to protect itself by different stresses, like excessive light, that induce its transition in hematocyst phase. In this phase the microalgae is more photoprotected and resistant to pigment photobleaching (Mascia and Girolomoni, 2017).

Large-scale microalgae cultivation systems exhibit photosynthetic conversion yield below 3% while the theoretical maxima of solar energy conversion into biomass for microalgae was calculated around 10-12% (Ooms et al., 2016). The lower yield is due to a series of bottlenecks as discussed in the following.
(i) Contamination of the cultivation systems by competing organisms including bacteria, yeast, fungi, weed algae/cyanobacteria, and protozoans. Biological contaminants must be quickly and effectively controlled, as these organisms can rapidly cause complete loss of production batches (Gonzalez-Morales et al., 2020).
(ii) A fraction of the photosynthetic active radiation (PAR) is not absorbable by a photosynthetic pigment (green and far-red light) and is thus wasted.
(iii) The high density of the cells inside a photobioreactor reduces light penetration in the inner layers.
(iv) Light that exceeds the saturation limit of the photosynthetic microorganisms is lost. Essentially, if more light energy is absorbed compared to the metabolic feasibility of light energy conversion, the energy absorbed in excess cannot be used for photochemistry but it is lost as heat. The heat dissipation of the excess energy absorbed can be a controlled active mechanism (called also Non-photochemical quenching, NPQ) or an uncontrolled reaction which leads to photoinhibition (Aro et al., 1993). The light saturation effect is evident comparing the photosynthetic response curves (Oz production or CO₂ consumption) with the light intensity given to microalgae (Li et al., 2009). In low light, photosynthetic rates increase linearly with the increasing irradiance, and the rate of photon absorption is determined by electron transport from water to CO₂. At higher irradiance the photosynthetic rate increases non-linearly with respect to light intensity till it reaches a light saturated region (Pmax) where photosynthetic rates are independent on irradiance. During the linear phase, light energy is efficiently funnelled to photosynthesis being used for the photochemical reactions, while at higher light CO₂ fixation and/or electron acceptors availability became the limiting step and the excess of light energy absorbed is not converted into chemical energy to produce new biomass. When light intensity is too high and electron transport is saturated, the excess energy is not converted to chemical energy but it increases the amount of chlorophyll (Chl) singlet excited states (¹Chl*) and the probability for population of chlorophyll triplet excited states (³Chl*), a species that reacts with molecular oxygen (O₂) to yield singlet oxygen (¹O₂). Because of its strong oxidizing potentials, ¹O₂ induces damage in its local environment by destroying lipids, nucleic acids and proteins yielding into widespread oxidation reducing biomass accumulation (Krieger-Liszkay, 2005). ROS generation is a constitutive and unavoidable process occurring in pigment-protein complexes upon illumination. Photodamage is not dependent only on the light intensity per se but also on its variation; sudden fluctuations in light intensity shortly overload photosystems with excess light energy generating ROS (Davis et al., 2016). It is dependent also on environmental and metabolic conditions that reduce the capacity of electron transport. Primary source of ROS and photoinhibition is PSII reaction centers. In excess light, electron flow cannot keep up with the charge separation, leading to increased lifetime of P680+ and singlet oxygen generation. PSII could also produce both superoxide anion (O-) and hydroxyl radical (OH·) in high light condition (Cleland and Grace, 1999, Pospisil et al., 2004). PSI is less affected because P700+ is far less oxidizing than P680+ and acts as a very efficient quencher but, when the NADPH pool is over-reduced, excess photo-excitation energy can reduce O₂, generating ROS, including superoxide anion radical (O₂-), hydrogen peroxide (H₂O₂), and hydroxyl radical (·OH) (Asada, 2006). ROS do not generate exclusively at the RC because also the chlorophylls bound to the light-harvesting complexes could become photosensitizers when the reaction centres are prolonged closed due to overexcitation (Horton, 2012). This would lead to chloroplast damage and decrease of photosynthesis yield if the damage overcame the rate of repair mechanisms.

Contaminant organisms have been recognized as one of the major constraint for large-scale cultivation of microalgae (including cyanobacteria), which occurs not only in open cultivation systems, but also on closed and hybrid systems (Wang et al., 2013, Gonzalez-Morales et al., 2020). To control contamination, chemical treatments such as use of herbicides, antibiotics, detergents, hypochlorite, and phenol are often used (Gupta et al., 2019, González-Morales et al., 2020). Moreover, in most cases sterilization of growth media and bioreactors must be implemented to maintain the desired monoculture. However, these practices substantially increase operating costs for closed or hybrid bioreactors and open ponds cannot be sterilized or kept under sterile conditions. A strategy to deal with contamination in outdoor open ponds is the use of selective culture environments such as high salt concentrations for halotolerant strains (Mendes and Vermelho, 2013, Sing et al., 2014, González-Morales et al., 2020) or N-deprived media for N-fixing cyanobacteria (Singh et al., 2016). Alternatively, fine control of pH has been proposed as a method for controlling bacterial contaminations (patent U.S. Pat. No. 9,181,523B1). Unfortunately, these practices are limited to a few species and can also affect final product quality. An additional measure which may favour the establishment of the target strain is using high inoculum percentage to allow the selected culture to dominate the system, even if this procedure is complicated at large-scale cultivation system. The use of bacterial phosphite dehydrogenase gene allowing to metabolize phosphite as phosphorous source has been proposed as a selection strategy for microalgae and cyanobacteria cultivation (González-Morales et al., 2020).

The primary reason that microalgae cannot be cultivated at a high photosynthetic efficiency under outdoor conditions is the non-homogeneous distribution of sunlight in photobioreactors. Cells near the reactor surface become oversaturated due to being exposed to the intense sunlight, resulting in dissipation of excess light energy. By improving the light energy distribution within the reactor, the effects of oversaturation are alleviated. To date, the concept of light dilution is a frequently employed approach to optimize photobioreactor performance by increasing its surface area per unit of reactor (Tredici and Zittelli, 1998, Dye, 2010, Cuaresma et al., 2011, Zemke et al., 2013) whereby the light use efficiency can be increased. However, physical and economic limits are being reached in the context of the hardware while there continues to be opportunities for improvement in relationship to biology.

An alternative approach to light dilution is to genetically engineer the microalgal cells to reduce the size of their antenna complexes (Mussgnug et al., 2005, Ort et al., 2011, Formighieri et al., 2012, Perrine et al., 2012, Kwon et al., 2013). Antennae are light-harvesting systems that are evident in all known photosynthetic organisms (Grossman et al., 1995). They comprise protein-pigment complexes located in, or on, photosynthetic membranes. With genetic modification, the pigment content of the antennae can be reduced. These so-called antenna mutants are more transparent and are light saturated at greater light intensities than wild-type cells. Antenna mutants absorb less light per cell, however, conversion of solar energy to chemical energy is expected to occur at a higher efficiency if direct sunlight is no longer oversaturating. Studies have demonstrated that cultures of antenna mutants at specific growth conditions have greater photosynthetic activity per unit of chlorophyll (Nakajima and Ueda, 2000, Kirst et al., 2012, Perrine et al., 2012, Cazzaniga et al., 2014, Kirst and Melis, 2014, Ort et al., 2015, Dall'Osto et al., 2019, Cecchin et al., 2020) (patent application WO2013063018A1). The possible application of these results for improving mass cultivation of microalgae are however still under debate (de Mooij et al., 2015).

Microalgae have different mechanism to protect from excess light energy. On the long term (hours-days), the control of light absorption occurs by regulation of chlorophyll content in cells and reorganization of photosystem architecture to reduce light absorption (Bonente et al., 2012). The size of photosynthetic antenna systems and the PSII/PSI ratio are reduced. Antioxidant molecules, like tocopherol, glutathione and ascorbate, and enzymes, like superoxide dismutase and ascorbate peroxidase, are accumulated. On the short term (seconds-minutes), non photochemical quenching (NPQ) allow the switch of the antenna of the photosystem from a light-absorbance state to a dissipative state where the excess light energy absorbed is emitted as heat avoiding the formation of excess ¹Chl* (Horton, 1996). NPQ is activated by acidification of the lumen, the inner compartment of thylakoid membranes in the chloroplast in a feedback-regulatory mechanism for excitation energy transfer to reaction centres. This regulation machinery ensures that quenching applies to the fraction of Chl excited states exceeding the capacity for use by the cell metabolism.

These mechanisms are efficient in avoiding photodamage and guarantee organism survival in natural conditions where they evolved but the condition of growth of microalgae inside bioreactors are far from these conditions. All these photoprotective mechanisms have evolved to be robust not efficient, they work to guarantee algae survival in natural condition not to reach maximal productivity (Zhu et al., 2010). In natural ambient microalgae are normally in low light condition because of the limited capacity of low-energy red radiation to penetrate water and the percentage of light reflected by the surface. The main light changes at which microalgae are exposed are the daily cycle of light and dark, the fluctuation of light intensities during the day and the seasonal oscillation of daylight length as a result of the rotation of the planet. In industrial algal cultivations, continuous illumination with light at saturating intensity is often used to maximize the biomass production. Moreover, algal growth is challenged by constantly changing irradiances: due to the mixing, cells rapidly move between layers of low vs. high illumination, which expose cells to far higher photodamage that the one they are used in natural condition. The same happens when algae are subject to cycles of dilution to recover the accumulated biomass. Strategies manipulate NPQ to improve biomass productivity has been reported for microalgae (WO2012092033A1, WO2017070404A2). Strategies directed at enhancing the resistance of microalgae to oxidative stress could increase their productivity; a Chlorella vulgaris mutant selected for an increased resistance to 102 improved the growth inside photobioreactor (Dall'Osto et al., 2019). Carotenoids that are involved both in NPQ and scavenging of ROS are a good target for improving microalgae resistance to light stress.

Astaxanthin has multiple purported health benefits on biological systems due to its action against ROS (Jyonouchi et al., 1995, Bennedsen et al., 1999). Astaxanthin has potential uses as an antitumor agent (Palozza et al., 2009, Zhang and Wang, 2015, Kim et al., 2016), the prevention of cardiovascular as well as neurological diseases, and diabetes (Uchiyama et al., 2002, Gross and Lockwood, Wu et al., 2015). Moreover, astaxanthin can be used as human dietary supplement and in aquaculture to improve fish colour (Hussein et al., 2006, Li et al., 2011, Yuan et al., 2011). Other ketocarotenoids like canthaxanthin, an intermediate of astaxanthin synthesis, has properties similar to astaxanthin, with high potential for use in human health applications (Miki, 1991, Møller et al., 2000). With few exceptions, higher-plants do not synthetize astaxanthin (Cunningham and Gantt, 2011), which is currently produced industrially from unicellular photosynthetic microalgae such as Haematococcus lacustris (recently renamed from H. pluvialis (Boussiba and Vonshak, 1991, Nakada and Ota, 2016)) or, to a lesser-extent, Chromochloris zofingiensis (Chen et al., 2017). The enzymes involved in astaxanthin synthesis are 3,3′-β-hydroxylase (crtz gene in microalgae) and 4,4′-β-ketolase (BKT, crto gene in microalgae) (Lotan and Hirschberg, 1995, Grossman et al., 2004). H. lacustris, is currently the main natural source of astaxanthin as it can accumulate to up to 90% of total carotenoids and 5% of cell dry weight (Bubrick, 1991) under certain environmental conditions. Astaxanthin accumulation in this alga is induced by stress conditions such as nitrogen or phosphorus starvation, high light, salt stress and elevated temperature (Boussiba and Vonshak, 1991) which stimulate the transition from motile zoospores (macrozooids) to immotile spores (aplanospores) (Kobayashi et al., 1997). These changes are accompanied by a degradation of the photosynthetic machinery and cessation of growth (Mascia et al., 2017) as well as the formation of thick and resistant cell walls (cysts) (Boussiba and Vonshak, 1991). The complexities of cellular changes to generate astaxanthin accumulation in H. lacustris requires a two-stage cultivation and results in a low overall productivity for the whole process. Moreover, the recalcitrance of aplanospore cell walls reduces the bio-accessibility of astaxanthin and makes mechanical disruption necessary in order to release astaxanthin for human or animal consumption (Kang and Sim, 2008), a process which increases production process costs.

Given these limitations, genetic engineering approaches have been undertaken to enable astaxanthin production in different biotechnological host organisms in order to generate suitable alternatives to traditional H. lacustris production processes. Astaxanthin synthesis has indeed been demonstrated in many different organisms such as fermentative bacteria (Henke et al., 2016, Park et al., 2018) as well as photosynthetic cyanobacteria (Harker and Hirschberg, 1997), and eukaryotic hosts including yeasts (Kildegaard et al., 2017), (Miura et al., 1998) and higher plants (Mann et al., 2000) (Stalberg et al., 2003) (Jayaraj et al., 2008) (Hasunuma et al., 2008) (Zhong et al., 2011) (Huang et al., 2013) (Harada et al., 2014, Nogueira et al., 2017) by the transgenic expression of keto- and hydroxylases. The results obtained were promising but with limited, industrial relevance due to the high costs of cultivation of these organisms and/or low productivity. Even if high production yields of astaxanthin have been reported upon heterotrophic cultivation of different microorganisms, the possibility to produce ketocarotenoids in photoautotrophic systems has a strong advantage in terms of sustainability, by consuming CO₂ and avoiding the costs of reduced carbon sources used in heterotrophic cultivation. In order to develop a sustainable alternative to traditional astaxanthin production, Chlamydomonas reinhardtii was engineered to constitutively produce astaxanthin and canthaxanthin (Leon et al., 2007, Tan et al., 2007, Zheng et al., 2014, Perozeni et al., 2020b). In the case of (Perozeni et al., 2020b) the C. reinhardtii strains resulting from insertion of the synthetic BKT gene, optimized for expression in C. reinhardtii, exhibited reddish-brown phenotypes, and reached astaxanthin productivities comparable to H. lacustris cultivation without many of its natural process constraints. The strong advantage of the efficient production of astaxanthin in C. reinhardtii was reported into the much weaker cell wall of this species, which makes the astaxanthin more bio-available for animal of human assimilation. The in vitro simulated digestion showed enhanced pigment extraction from C. reinhardtii compared to H. pluvialis, indicating that the engineered alga can be used directly in aquaculture and nutraceutical feed, without the need for prior pigment extraction (Perozeni et al., 2020b).

### SUMMARY OF THE INVENTION

It is an object of the present invention to provide the use of a transgene and corresponding protein product that allows to improve the biomass production of microalgae at high irradiance and, possibly, also prevent growth of competing contaminants, in particular other photosynthetic organisms.

This object is achieved by means of the use of a polypeptide or nucleic acid as defined in claim 1.

### BRIEF DESCRIPTION OF THE DRAWINGS

FIG. 1 shows the characterization of (A, B) PSII operating efficiency (estimates the fraction of PSII centres with reduced QA), (E, F) relative electron transport rate (ETR), and (G, H) photosynthetic O₂ evolution at different actinic light intensities for Chlamydomonas reinhardtii background strain (BS) (square) and BKT-expressing cells (BKT, circle) cells adapted in control (CL left) of high (HL right) light. Net photosynthetic rate data were fitted with Hill equation. Data are expressed as mean±SD. n>3. * indicate BKT values that are significantly different (Student's test, P<0.05) from BS.

FIG. 2 shows oxygen evolution during high light exposure. In FIG. 2A, BS (black) and BKT (grey) cells were illuminated with cycles of five minutes of illumination at 6000 μmol photons m⁻2s⁻¹ (light bar above graph) and three minutes of dark (black bar above graph) and oxygen evolution was recorded. FIG. 2B shows oxygen evolution during continuous illumination of BS (black) and BKT (grey) strains with 6000 μmol photons m⁻²s⁻¹.

FIG. 3 shows the nonphotochemical quenching (NPQ) phenotype of BKT expressing lines as compared to control cell lines. In particular, FIGS. 3A and 3B show graphs of the measurement of NPQ kinetics on BS (line with squares), BKT (line with circles) and npq4 lhcsr1 (npq4.1, line with triangles), cells using actinic lights of 1,200 μmol photons m-2s-1. FIGS. 3C and 3D show graphs of the NPQ value after 10 min of illumination with different actinic light intensities. Cells were adapted in control (CL left) or high light (HL right). Data are expressed as mean±SD (n=4). * indicate BKT values that are significantly different (Student's t test, P<0.05) from BS.

FIG. 4 shows photooxidation of C. reinhardtii cells under photoxidative stress. In FIG. 4A, BS (line with squares) and BKT (line with circles) cell suspension were treated with strong white light (14,000 μmol photons m-2s-1, 20° C.) and the decrease of Chl was evaluated by measuring the absorption area in the region 620-740 nm. Data are expressed as mean+SD (n=4). In FIG. 4B, BS (line with squares) and BKT (line with circles) cell suspension, incubated with Singlet Oxygen Sensor Green (SOSG), were illuminated with 2000 μmol μmol photons m⁻²s⁻¹ at 20° C. Singlet oxygen production was measured as increased fluorescence emission of SOSG with respect to the initial value (excitation 480 nm, emission 510-540 nm). Data are expressed as mean±SD (n=2). * indicate BKT values that are significantly different (Student's t test, P<0.05) from BS.

FIG. 5 shows growth curves of BS and BKT. In particular, FIGS. 5A to 5D show growth curves of BS (black) and BKT (grey) cultivated with 3% CO₂ at 100 and 3000 μmol photons m⁻²s⁻¹ in HS or TAP medium. At 3000 μmol photons m² s⁻¹ cell were manually diluted to 0.1 OD when the stationary phase was reached. FIGS. 5E and 5F show volumetric maximal productivity calculated from the growth curve in HS (E) or TAP (F).

FIG. 6 shows a titration curve of BKT cells in competitive growth. At the end of the experiment described in FIG. 5 different percentage of BKT cells, from 0 to 100%, were added to BS cells. Astaxanthin content, in the different combination, was quantified by fitting of the acetone extract in order to create the correlation (linear fitting) between astaxanthin amount and BKT cells percentage. This correlation was used to estimate, from the astaxanthin content, the percentage of BKT cells in mix tube of the competitive growth shown in FIG. 7.

FIG. 7 shows competitive growth of BS and BKT strains. In FIG. 7A, cell suspensions, containing 1*10⁶ cell/ml of BS, BKT or a mix of the two genotypes in equal amount, were grown at 3000 μmol photons m⁻²s⁻¹ for three days and pictures were taken, at the end of the three days. In FIG. 7B, a picture was taken at the end of the co-cultivation of BKT with C. vulgaris (Cv). From left to right: culture starting at the beginning of the growth phase at 1*10⁶ cell/ml of BKT, culture starting from 1*10⁶ cell/ml of C. vulgaris (Cv) and two replica of mix tube starting with same amount of the two algae at a cell ratio 1:1. The inset shows the calculated percentage of BKT cells inside the mix tube. FIG. 7C shows the same set-up as FIG. 7B, except that in this case C. vulgaris (Cv) colture started from the amount of cells that gave the same absorption area (650-730 nm) as 1*10⁶ cell/ml of BKT. The mix tubes started with cells of BKT and C. vulgaris in a 1:1 ratio on the base of the cell absorption in the 650-730 nm range. FIG. 7D shows acetone spectra of the pigments extracted from the tubes of BS (black), BKT (grey) and the mix (dotted line) end of experiment; spectra are normalized to the qY absorbance. The insets show the calculated percentage of BKT cells inside the mix tube. FIG. 7E shows the same experiment repeated in HS medium.

FIG. 8 shows the growth curve of a Synechococcus PCC 11901 strain engineered in order to produce astaxanthin (BKT) compared to its background strain (BS). The BKT strain was obtained by overexpression of BKT and CrtZ enzymes. The expression of the latter is usually low in cyanobacteria, differently from green algae, and its overexpression is necessary to induce the high astaxanthin accumulation observed in the BKT strain from C. reinhardtii (>50% of total carotenoids). The growth curve is reported as optical density at 720 nm. Cultivation was performed at 3% CO₂ and 2000 μmol m-2 s-1.

### DETAILED DESCRIPTION OF THE INVENTION

The present invention provides solutions for conferring a dominant growth feature to algal strains in mass culture.

More specifically, the present invention relates to the use of a polypeptide comprising SEQ ID NO:1, a sequence of the BKT polypeptide modified so as to optimize the amino acids (aa) codon usage and spreading of the RuBisCO small subunit II (rbcs2) intron 1 sequence in order to minimize exon lengths to enable robust transgene expression. In addition, the 116 aa C-terminal tail was omitted since its absence in BKT from other organisms and its expression in vitro demonstrated that is not necessary for its activity.

The expression of the BKT enzyme can be performed by several strategies. A preferred strategy is disclosed in the following, however the BKT polypeptide can be fused with other proteins, such as fluorescent proteins, or other tags or other proteins to confer the dominant growth feature in high light conditions.

In addition, efficient BKT expression can be obtained by inhibition of its mRNA degradation or BKT protein proteolysis to confer the dominant growth feature in high light conditions.

The polypeptide for use in the present invention preferably comprises SEQ ID NO: 2, which includes a sequence of the yellow fluorescent protein (YPF) fused to the BKT polypeptide. More preferably, the polypeptide comprises SEQ ID NO: 3, which further comprises the PsaD transit peptide, which allows to localise the peptide into the chloroplast. Even more preferably, the polypeptide comprises SEQ ID NO: 4, further including the Strep-tag II sequence which results in orange/red colonies.

The nucleic acid for use according to the present invention is a nucleic acid encoding for the above said polypeptides. It therefore comprises SEQ ID NO: 5 (CrBKT sequence), preferably SEQ ID NO: 6 (CrBKT YPT sequence), more preferably SEQ ID NO: 7 (CrBKT_YFP+PsaD sequence), even more preferably SEQ ID NO: 8 (CrBKT_YFP PsaD+Strep-tag II sequence). The nucleic acid even more preferably comprises the sequence for paromomycin or spectinomycin to select transformant lines using antibiotic resistance.

The present invention also relates to the use of expression vectors comprising the above said nucleic acid sequences. The pOpt2_mVenus Paro vector is preferred. This expression vector can be purchased at the international center for Chlamydomonas research (https://www.chlamycollection.org).

The polypeptide for use in the present invention is preferably expressed in a cell of a unicellular photosynthetic organism. The unicellular photosynthetic organism is preferably a microalgae, more preferably of the genus Chlamydomonas, even more preferably of the species reinhardtii.

The above said polypeptides, nucleic acids or expression vectors are used for improving the resistance to oxidative stress and/or photoinhibition of host organisms or for improving biomass productivity of host organisms and/or prevailing over other competing organisms upon cultivation in high light conditions and in the presence of at least 1% CO₂.

It is well known (see for instance Cecchin et al 2021 https://onlinelibrary.wiley.com/doi/10.1111/pce.14074) that CO₂ at atmospheric concentration (0.04%) is not sufficient for the high photosynthetic activity of microalgae. In the examples that follow cells were cultivated at 3% CO₂: at this concentration the carbon availability allows to properly exploit the increased photosynthetic efficiency and biomass productivity of BKT expressing strains. The carbon concentration in order to prevent carbon limitation depends on the specific species cultivated, but it is widely recognized in the literature that a CO₂ concentration higher than 1% provide sufficient carbon to avoid carbon limitation. In some cases, CO₂ concentration can even reach 10 to 15%.

Microalgae strains, in particular C. reinhardtii strains, engineered with BKT enzymes display several positive features that lead to improve biomass production:
1) reduced chlorophyll content (around 90%) making this strain a “pale green” strain;
2) strongly reduced non-photochemical quenching (NPQ) of the light absorbed, indicating that heat dissipation of light energy absorbed is strongly reduced and that the photochemical reaction are rather favoured, leading to generation of chemical energy required for CO₂ assimilation;
3) high accumulation of astaxanthin, which confers a strong antioxidant activity preventing photoinhibition.

The advantage provided by these combined features for cultivation of microalgae in high light conditions were totally unexpected. As a matter of fact, similar chlorophyll/cell reduction and high astaxanthin accumulation were reported for H. lacustris (formely H. pluvialis) under high light conditions leading however to a strong reduction in growth rate (Fan et al., 1998, Scibilia et al., 2015). Similarly, when astaxanthin biosynthesis was induced in higher plants, a reduced or similar photosynthetic activity and growth was observed (Hasunuma et al., 2008, Röding et al., 2015, Fujii et al., 2016).

Genetic engineering of microalgae strains, in particular C. reinhardtii, for optimized expression of BKT enzyme used according to the invention allows the target strain to grow efficiently in high light (above 1000 μmol m-2 s-1), being more resistant to oxidative stress and being able to efficiently use the light energy available for photosynthesis.

Co-cultivation of the BKT-expressing strains used according to the invention with other microalgae strains at strong light allows a selective growth of BKT-expressing strains. The advantages of the present invention are therefore to 1) improve biomass and metabolites productivity making the engineered strains capable of using more efficiently light energy available; 2) implement a selection strategy for target strains as for instance strains previously engineered for producing of desired metabolic products reducing the risk of growth of other photosynthetic contaminant microorganisms.

The fast growth observed for BKT-expressing strains used according to the present invention also allowed to grow them in unsterile conditions in high light conditions (3000 μmol m⁻² s⁻¹) where the control strains were heavily contaminated by other microorganisms.

The cultivation of BKT-expressing lines can be performed in high light in closed photobioreactors or open ponds or hybrid systems.

The nutrient solution used for the cultivation of strains can vary as compared to the solution used in the examples, using ammonia or nitrate as nitrogen source, phosphate or phosphite as phosphorous source with a specific composition of macro- and micronutrients optimized for microalgae growth.

The BKT gene can be introduced and expressed in other microalgal or cyanobacteria strains with respect to that used in the examples in order to confer the dominant growth feature in high light conditions.

The definition of “high light” for the application of this invention depends on specific photosynthetic features of the species considered. On the basis of the oxygen evolution curve at different light intensities, it is possible to define a “high light” condition for the specific species considered an irradiance above the saturation limit (where the dependency between net oxygen evolution rate and light intensity given to the cells is not linear). In particular, “high light” corresponds to the light intensity measured as μmol m-2 s-1 at which a specific photosynthetic species exhibits a saturation of its photosynthetic activity measured following the light dependent oxygen evolution curves.

Cultivation of the host organism occurs preferably with 2-4% CO₂, more preferably with about 3% CO₂.

### EXAMPLES

BKT expressing strains were obtained transforming C. reinhardtii with the optimized version of the BKT gene used according to the invention (SEQ ID NO: 8 in particular). The selection of the trasformant lines was done by using an antibiotic resistance present in the construct used for transformation (paromomycin or spectinomycin). Selection of BKT expressing colonies was done on the base of orange/red color of the colonies.

### Example 1—Pigments Composition

BKT-expressing lines were adapted for two weeks photoautotrophically in high-salt (HS) minimal media (Harris and Harris, 2008) at two different light intensity: control (CL, 80-100 μmol photons m² s⁻¹) and high light (HL, 400-600 μmol photons m² s⁻¹). Pigments were then extracted in acetone and analysed by spectral absorbance (Table 1). BKT-expressing lines were able to accumulate a high amount of ketocarotenoids; more than 50% of carotenoids were converted in ketocarotenoids in CL and this percentage rise to 59% in higher light. Chl content in the BKT-expressing lines: at CL chlorophyll content per cell was decreased by 30% compared to the parental strains. In cells adapted to higher light the difference is higher with a decrease of the 50% in BKT expressing lines.

High-performance liquid chromatography (HPLC) of the acetone extract allowed identifying and quantifying single carotenoids and confirmed that ketocarotenoids were present in BKT-expressing lines. The HPLC showed that astaxanthin is the main carotenoid accumulated with lower amount of canthaxanthin, adonirubin and adonixanthin while hydroxy-echinenone was detected only in minimal traces. In HL there was also an increase of the amount of canthaxanthin. The distribution of carotenoids, normalized to chl, showed that in BKT-expressing lines there was a relative decrease of all the carotenoids normally present in C. reinhardtii. In CL the decrease was higher for the β-β xanthophylls neoxanthin and violaxanthin (in CL zeaxanthin was accumulated only in low amount) that were reduced of the ˜ 70% and 80% respectively while lutein and β carotene were reduced of the 20%. In HL these carotenoids were reduced to ˜ 50% of the value in the BACKGROUND STRAIN (BS) with violaxanthin reduced to 20%.

Table 1 shows pigment content and Fv/Fm of BS and BKT. Pigments content determined in cells grown at control (CL) or high (HL) light in HS 1 week starting from 5*10⁵ cells/ml. Data are expressed as means±SD (n=4). * indicates BKT values that are significantly different (Student's t test, P<0.05) from BS. Abbreviation indicate: Chlorophyll (chl), total carotenoid (car), total ketocarotenoid (keto).

| | | pg | | | % | |
|---|---|---|---|---|---|---|
| | | chl/cell | chl a/b | chl/car | keto/car | Fv/Fm |
| CL | BS | 3.14 ± 0.34 | 2.59 ± 0.06 | 3.2 ± 0.04 | 0 ± 0 | 0.76 ± 0.01 |
| | BKT | 2.09 ± 0.21* | 2.46 ± 0.06* | 2.74 ± 0.06* | 51.07 ± 2.90* | 0.65 ± 0.02* |
| HL | BS | 2.34 ± 0.03 | 2.74 ± 0.02 | 2.62 ± 0.04 | 0 ± 0 | 0.72 ± 0.02 |
| | BKT | 1.28 ± 0.06* | 2.52 ± 0.05* | 2.18 ± 0.05* | 58.61 ± 0.80* | 0.52 ± 0.04* |

### Example 2—Photosynthetic Activity

Photosynthetic efficiency of BKT-expressing lines compared to the background strain was analyzed by fluorescence analysis using a Pulse Amplitude Modulated (PAM) fluorometer. Fluorescence induction in dark-adapted cells (Butler, 1973) revealed a significant decrease of PSII maximal efficiency (Fv/Fm) compared to the background strain (Table 1). Cells adapted to CL had an Fv/Fm of 0.76 in the background strain and of 0.65 in the BKT-expressing lines.

In HL there is a decrease of the value in the background strain to 0.72 but the decrease is higher in BKT-expressing lines that had an Fv/Fm reduced to 0.52. At 100 μmol photons m² s⁻¹ BKT-expressing lines had a lower PSII operating efficiency and relative electron transport rate (ETR) with respect to the background strain but at the higher lights the mutant showed a value identical or higher than wild type.

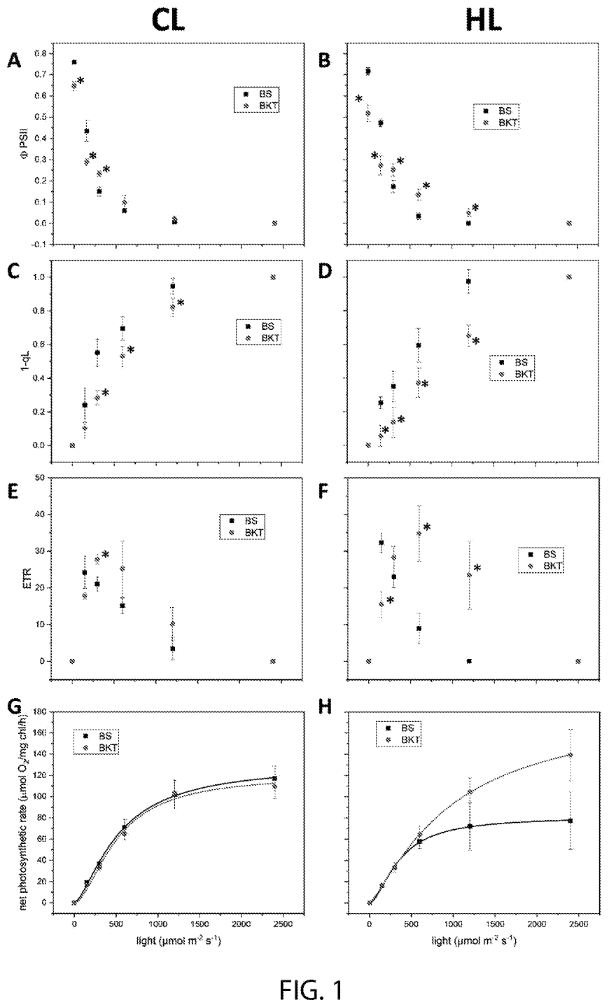

In cells adapted to HL these two parameters were still lower at 100 μmol photons m² s⁻¹ but at all the higher light tested the mutant showed and improved PSII efficiency and a higher electron transport. The QA reduction, measured as 1-qL, is lower in the mutant in cells adapted both in LL or HL. The difference in PSII activity was evident also by the photosynthetic light curves (FIGS. 1A to 1H). In cell adapted in low light, BKT-expressing lines and the background strain showed similar maximum level of oxygen evolved (Pmax), half-saturation light intensity and slope of linear phase of light dependent increase. In HL the slope of linear increase is still similar but the BKT-expressing lines reached and higher Pmax and the photosynthesis is saturated at a higher light intensity: the half-saturation light intensity is ˜ 350 μmol photons m² s⁻¹ for the background strain and 900 μmol photons m² s⁻¹ for BKT-expressing lines. The PSI yield and ETR, measured at the same light intensities used for PSII, showed similar value for the background strain and BKT-expressing lines.

These data indicate that the mutant accumulating ketocarotenoid had a better PSII efficiency in high condition where photoinhibiton affects photosynthesis.

### Example 3—Resistance to Photoinhibition, Role of NPQ and ROS Scavenging

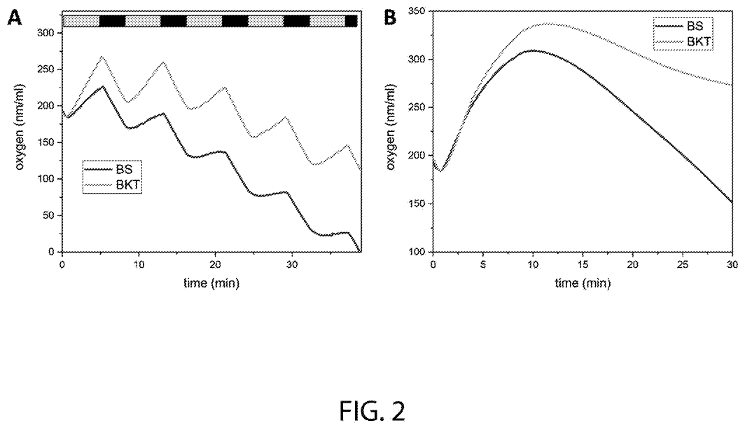

The resistance to photoinhibition and photodamage due to excess light was analysed on the two strains. Cells of the background strain and BKT-expressing lines were illuminated with cycles of five minute of strong light (6000 μmol photons m² s⁻¹) and three minutes of dark while oxygen evolution was registered (FIG. 2a). FIG. 2 shows that oxygen evolution decreases with every cycle because of light-dependent photoinhibiton but the effect is stronger in the background strain where, after five cycles of illumination, oxygen production was around 20% of the first cycle rate while the BKT-expressing lines maintain 70% of its initial rate. Oxygen evolution was also monitored during continuous illumination with the same strong light (6000 μmol photons m⁻²s⁻¹ for thirty minutes (FIG. 2B). In the first minutes, the oxygen rate increased linearly then the slope diminished until it reached a plateau after which the oxygen evolution decreased for the effect of photoinhibition and of the shutting down of PSII oxygen evolving complex. BKT-expressing lines reached a higher level of oxygen production and the successive decrease is slower. After 30 minutes of illumination the oxygen evolution in the mutant tend to stabilize to a plateau while the background strain rapidly falls down toward zero.

These experiments confirm that BKT-expressing lines are and preserve their more resistant to photoinhibiton photosynthetic activity upon illumination to strong light.

### Example 4—Nonphotochemical Quenching (NPQ)

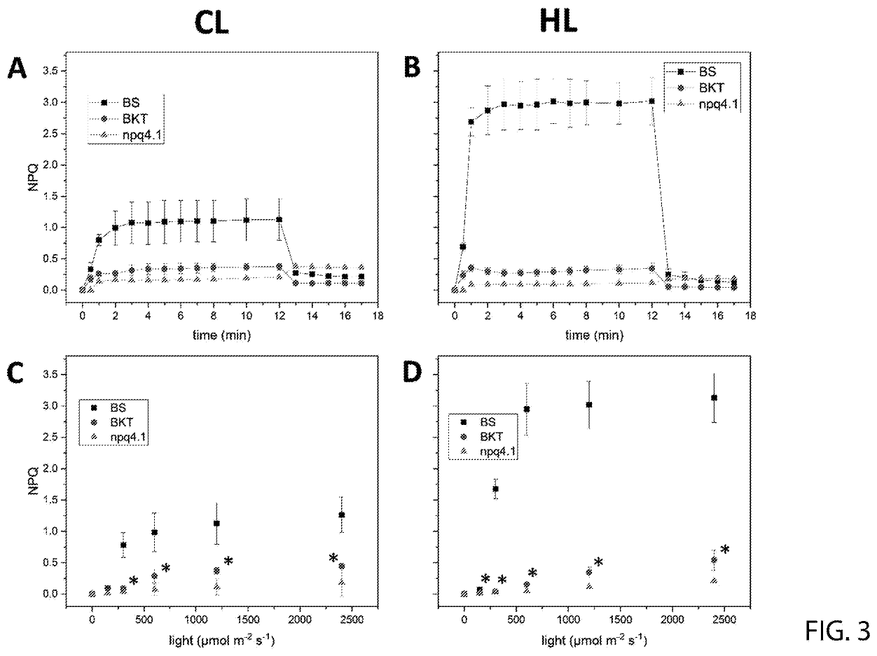

One of the major mechanisms for photoprotection is NPQ that was then measured (FIG. 3) (Horton, 1996). In this set of measures, the double mutant npq4 lhcsr1 was also added. This mutant is unable to activate NPQ as negative control (Ballottari M. et al., 2016). At the different conditions tested, the NPQ phenotype of BKT expressing lines was essentially similar to the npq4 lhcsr1 case, demonstrating that this photoprotective mechanisms was almost not activated in BKT engineered lines. The stronger resistance to photoinhibiton of the BKT-expressing lines was independent from the NPQ and it cause lied somewhere else. BKT lines rather exhibit a reduced heat dissipation of the light energy absorbed, allowing for a higher energy availability for photochemical reactions.

### Example 5—Photooxidation of BKT Under Photoxidative Stress

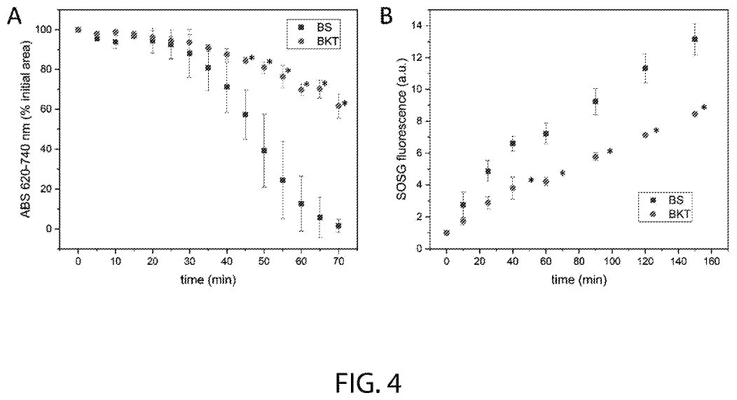

Another possible reason for the improved photoresistance of BKT-expressing lines was the higher antioxidant activity of ketocarotenoid and their ability to work as sunscreen for photosynthetic pigments. To check this hypothesis, we proceed to monitor Chl bleaching and ROS production during illumination with strong high light. Cells of the background strain and BKT-expressing lines were illuminated with a bleaching light and every five minutes chlorophyll absorption were registered (FIG. 4a). In the background strain there was a strong reduction of the absorbance and in seventy minutes Chl are completely bleached while, in the same time, in the BKT-expressing lines there was a reduction of absorbance of only 30% of the initial area. Then we proceeded to measure if this photoprotection correlated with a diminished production of ROS in the mutant thanks to the higher antioxidant activity of astaxanthin (FIG. 4b). Singlet oxygen generation was monitored, using sensor green during illumination of the cells with red high light. Sensor green is a fluorescent dye that increase is fluorescence after interaction with singlet oxygen and can be used to quantify singlet oxygen generation. This experiment showed a higher generation of singlet oxygen in the background strain with respect to BKT-expressing lines; the control had an increase of sensor green fluorescence that was almost the double with respect to BKT-expressing lines.

### Example 6—Biomass Productivity of BKT Expressing Lines

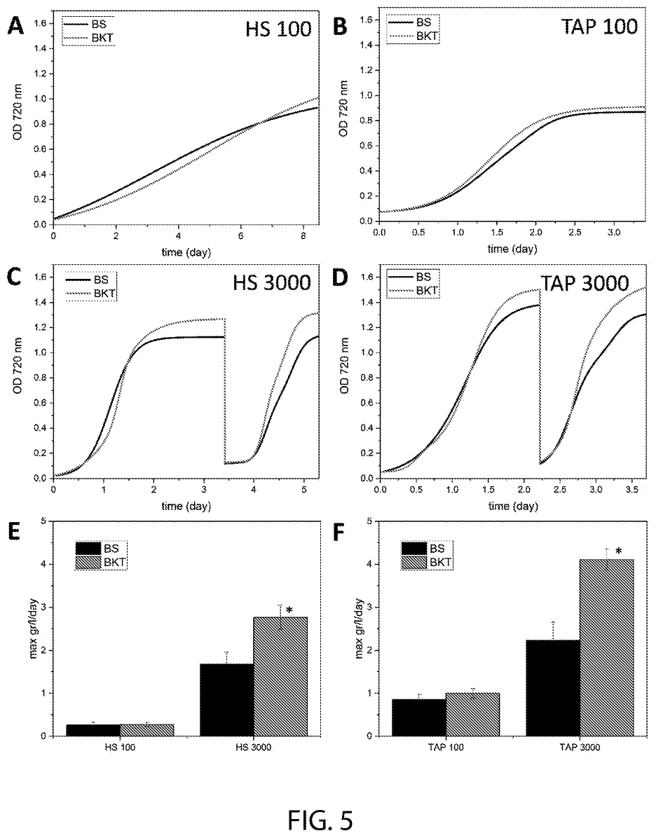

Biomass productivity and growth phenotype of BKT expressing lines was characterized in in 80 ml closed photobioreactors with LED illumination, in autotrophic (minimal HS medium) or mixotrophic (acetate supplied TAP); irradiance used for growth were 100 μmol photons m² s⁻¹ or at very high light at 3000 μmol photons m² s⁻¹. At 100 μmol photons m² s⁻¹ in HS and TAP media, biomass productivity, measured as gr/l/day, was similar between the two genotypes (FIG. 5). At 3000 μmol photons m² s⁻¹ BKT-expressing lines grow faster than the background strain with the maximal productivity is increased in the mutant (2.8 gr L⁻¹ day⁻¹ in BKT-expressing lines vs 1.8 gr L⁻¹ day⁻¹ vs. in the background strain). In mixotrophic condition (TAP medium), the BKT-expressing lines mutant grown faster and reached a higher OD with respect to the background strain from the first cycle, the average and maximal productivity were both higher in the mutant with respect to the control (4.1 gr L⁻¹ day⁻¹ in BKT-expressing lines vs. 2.2 gr L⁻¹ day⁻¹ in the background strain).

### Example 7—BKT as a Selective Trait for Growth in High Light

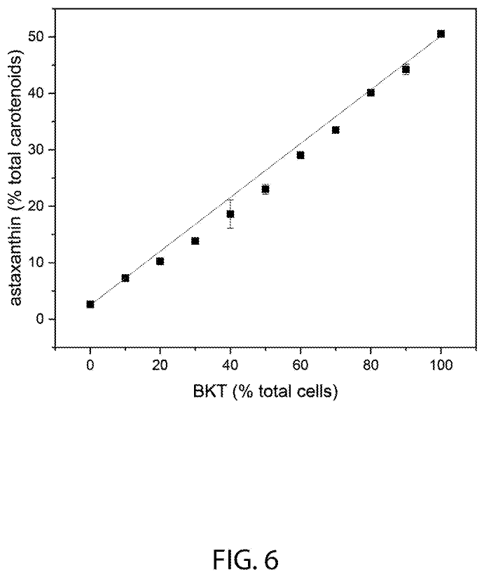

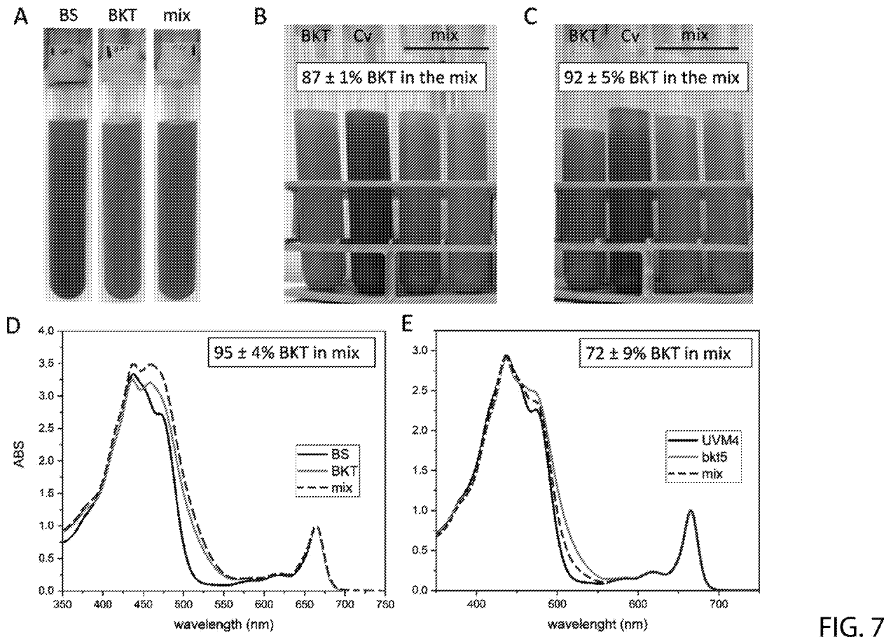

To further confirm this enhanced productivity of the BKT-expressing lines in high light, a “competition test” was performed. An equal amount of the background strain and BKT-expressing lines cells was added to same tube and illuminated with 3000 μmol photons m² s⁻¹ and after three days the content of the tube was analysed in order to see which genotype was more accumulated. Ketocarotenoids, with shifted their absorption, were present only in the BKT-expressing lines and extracting the pigments at the end would allow to determine the relative abundance of the two genotypes. In order to do this, tube with only the background strain and BKT-expressing lines cells were illuminated in parallel at the mixed tube as control. At the end of the experiment, cells from the control tubes were mixed in different amount and the pigments extracted to obtain calibration curve of genotype percentage vs ketocarotenoid absorbance (FIG. 6). First the test was done in TAP medium and the result was surprising: after three days more than 90% of the cells in the tube where BKT-expressing lines (FIG. 7). This could be clearly seen form the spectra of the cells at the end of the growth where BKT-expressing lines and mix tubes had the same shoulder at 530 nm that was absent in the background strain. The better performance of BKT-expressing lines was also evident from the colour of the tubes at the end of the experiment; the background strain had a bright green colour while the mix tube was orange like the BKT-expressing lines. The competition test was then repeated in HS medium; in this case, considering the lag phase in first cycle of growth of BKT-expressing lines compared to the background strain, tubes of the two genotypes were illuminate with 3000 μmol photons m² s⁻¹ till they reached plateau and then the same amount of the background strain and BKT-expressing lines were mixed and the test started. BKT-expressing lines showed a better performance in high light also in minimal medium and at the end of the experiment 75% of cells inside the tubes were BKT-expressing lines.

A similar competition test was also performed between BKT-expressing lines of C. reinhardtii and the microalga species Chlorella vulgaris, one of the fastest growing species known from the literature (Garcia-Cubero et al., 2018, Bernaerts et al., 2019, Cecchin et al., 2019). When C. vulgaris and BKT-expressing lines were cultivated at 3000 μmol photons m² s⁻¹ alone the biomass productivity was higher in the case of BKT-expressing lines. When the two strains were co-cultivated in 1:1 ratio on the base of cell number or absorption in the 600-750 nm light range, in both cases BKT-expressing line were dominant (>80%) at the end of the growth curve.

These competition tests, clearly point out how the expression of BKT enzyme and the presence of astaxanthin in C. reinhardtii increase its resistance to ROS and photoinhibiton, improve the performance in photobioreactor in high light and biomass accumulation, making BKT-expressing strains dominant.

### Example 8—the Expression of the Optimized Version of the BKT Gene in Synechococcus Leads to the Production of Astaxanthin and Accelerated Growth in High Light

This example is provided to prove that the optimized version of the BKT gene leads to similar effects in other microalgae strains. In particular, the case of the cyanobacterium Synechococcus PCC 11901 is reported.

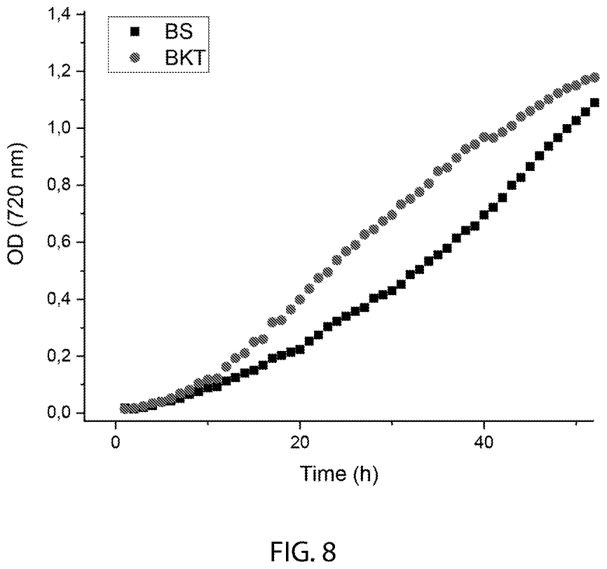

This strain does not produce astaxanthin and ketocarotenoids, since the BKT gene is absent in its genome. Synechococcus PCC 11901 was engineered inserting in its genome the CrBKT gene optimized for expression in this specific host. In particular, a previously known strong and constitutive promoter (Pcpt) was used to drive gene expression (Włodarczyk A, Selão TT, Norling B, Nixon PJ. Newly discovered Synechococcus sp. PCC 11901 is a robust cyanobacterial strain for high biomass production. Commun Biol. 2020 May 7; 3 (1): 215. doi: 10.1038/s42003-020-0910-8). Codon usage was optimized for Synechococcus and the transgene was inserted into the expression cassette by homologous recombination in the acsA locus, according to literature (Wlodarczyk et al., 2020). Differently from C. reinhardtii, in Synechococcus PCC 11901 also the CrtZ gene encoding for expressed at high level in a hydroxylase (already Chlamydomonas reinhardtii) was overexpressed in order to provide sufficient substrates for BKT enzymes. Indeed, in order for BKT to produce astaxanthin, a sufficient amount of substrate must be available. The substrate is provided by the enzyme hydroxylase, which in wild-type cyanobacteria is expressed at a low level. The hydroxylase therefore needs to be overexpressed. As in the case of Chlamydomonas reinhardtii, the percentage of astaxanthin per total carotenoids was higher than 50% in the engineered Synechococcus PCC 11901, topping 85%. As in the case of BKT expressing strain of c. reinhardtii, engineered Synechococcus PCC 11901 (producing astaxanthin) was characterized by a fastest growth in high light compared to its background (without astaxanthin, see FIG. 8).

1. A method for improving the resistance to oxidative stress and/or photoinhibition of host organisms or for improving biomass productivity of host organisms and/or prevailing over other competing organisms, wherein the host organism expresses a polypeptide comprising SEQ ID NO:1, or a nucleic acid encoding for the polypeptide comprising SEQ ID NO:1, or an expression vector comprising a nucleic acid encoding for the polypeptide comprising SEQ ID NO: 1 and is cultivated in high light conditions and in the presence of at least 1% CO₂.

2. The method according to claim 1 wherein the polypeptide comprises SEQ ID NO:2, SEQ ID NO:3, or SEQ ID NO:4.

3. The method according to claim 2 wherein the nucleic acid encodes for the polypeptide comprising SEQ ID NO:2, SEQ ID NO:3, or SEQ ID NO:4.

4. The method according to claim 3 wherein the nucleic acid encodes for the polypeptide comprising SEQ ID NO:5.

5. The method according to claim 3 wherein the expression vector comprises the nucleic acid encoding for the polypeptide comprising SEQ ID NO: 5.

6. The method according to claim 5, wherein the vector is pOpt2_m Venus_Paro.

7. The claim 1, wherein of the host organism is a unicellular photosynthetic organism.

8. The method according to claim 7, wherein the unicellular photosynthetic organism is a microalgae.

9. The method according to claim 8, wherein the microalgae is of the genus Chlamydomonas.

10. The method according to claim 9, wherein the Chlamydomonas cell is of the species reinhardtii.

11. The method according to claim 1, wherein high light conditions is >1000 μmol m-2 s-1.

12. The method according to claim 11, wherein high light conditions is >1500 μmol m-2 s-1.

13. The method according to claim 12, wherein high light conditions is >3000 μmol m-2 s-1.

14. The method according to claim 1, wherein cultivation is in the presence of 2-4% CO₂.

15. The method according claim 14, wherein cultivation is in the presence of about 3% CO₂.

16. The method according to claim 1, wherein at least one additional polypeptide encoding an enzyme involved in the biosynthesis of carotenoids or a nucleic acid encoding for the additional polypeptide is used.
